(19) United States
(12) Patent Application Publication
(10) Pub. No.: US 2012/0083668 A1
(43) Pub. Date: Apr. 5, 2012

(54) SYSTEMS AND METHODS TO MODIFY A CHARACTERISTIC OF A USER DEVICE BASED ON A NEUROLOGICAL AND/OR PHYSIOLOGICAL MEASUREMENT

(76) Inventors: Anantha Pradeep, Berkeley, CA (US); Ramachandran Gurumoorthy, Berkeley, CA (US); Robert T. Knight, Berkeley, CA (US)

(21) Appl. No.: 13/249,512

(22) Filed: Sep. 30, 2011

Related U.S. Application Data
(60) Provisional application No. 61/388,495, filed on Sep. 30, 2010.

(51) Int. Cl. A61B 5/00

(52) U.S. Cl. 600/300

(57) ABSTRACT

Example methods, systems and tangible machine readable instructions to operate a user device are disclosed herein. An example method of operating a user device includes collecting at least one of neurological data or physiological data of a user interacting with the user device. The example method also includes identifying a current user state based on the at least one of the neurological data or the physiological data. In addition, the example method includes modifying a characteristic of the user device based on the current user state and a desired user state.

### RELATED APPLICATION

This patent claims the benefit of U.S. Provisional Patent Application Ser. No. 61/388,495, entitled “Intelligent Interfaces Based on Neurological and Physiological Measures,” which was filed on Sep. 30, 2010, and which is incorporated herein by reference in its entirety.

### FIELD OF THE DISCLOSURE

This disclosure relates generally to user devices, and, more particularly, to systems and methods to modify a characteristic of a user based on a neurological and/or physiological measurement.

### BACKGROUND

User devices such as mobile phones, televisions, computers, tablets, etc. are used in a variety of contexts including computing, business, training, simulation, social interaction, etc. User devices include user interfaces that are typically designed to be appealing to a user, easy to manipulate and customizable. However, traditional user devices and the associated user interfaces are typically limited in capability, adaptability, and intelligence.

### BRIEF DESCRIPTION OF THE DRAWINGS

FIG. 1A is a schematic illustration of an example system to modify a characteristic of a user device based on a neurological and/or physiological measurement.

FIG. 1B is a schematic illustration of an example apparatus to modify a characteristic of a user device based on a neurological and/or physiological measurement.

FIGS. 2A-2E are schematic illustrations of an example data collector for use with the example system of FIG. 1A and/or the example apparatus of FIG. 1B.

FIG. 3 is a flow chart representative of example machine readable instructions that may be executed to implement the example system of FIG. 1A, the example apparatus of FIG. 1B and/or the example data collector of FIGS. 2A-2E.

FIG. 4 illustrates an example processor platform that may execute the instructions of FIG. 3 to implement any or all of the example methods, systems and/or apparatus disclosed herein.

### DETAILED DESCRIPTION

Example customizable, intelligent user devices including user interfaces are disclosed herein that have operating characteristics that are dynamically modified based on user neurological and/or physiological states. Example interfaces include, for example, an interface for a computer system, a business transaction device, an entertainment device, a mobile device (e.g., a mobile phone, a personal digital assistant), etc. In some examples, an operating characteristic of a user device is dynamically modified as changes in a measured user state reflecting attention, alertness, and/or engagement are detected. In some such examples, user profiles are maintained to identify characteristics of user devices including characteristics of user interfaces that are most effective for groups, subgroups, and/or individuals with particular neurological and/or physiological states or patterns. In some such examples, users are monitored using any desired biometric sensor. For example, users may be monitored using electroencephalography (EEG), cameras, infrared sensors, interaction speed detectors, touch sensors and/or any other suitable sensor. In some examples disclosed herein, configurations, fonts, content, organization and/or any other characteristic of a user device are dynamically modified based on changes in one or more user(s)' state(s). For example, biometric, neurological and/or physiological data including, for example, eye-tracking, galvanic skin response (GSR), electromyography (EMG), EEG and/or other data, may be used to assess an alertness of a user as the user interacts with the user device. In some examples, the biometric, neurological and/or physiological data is measured, for example, using a camera device associated with the user device and/or a tactile sensor such as a touch pad on a device such as a computer, a phone and/or a tablet.

Based on a user's state as indicated by the measured biometric, neurological and/or physiological data, one or more aspects of a disclosed example device are modified. In some examples, based on a user's state (e.g., the user's alertness level and/or changes therein), a font size and/or a font color, a scroll speed, an interface layout (including, for example showing and/or hiding one or more menus) and/or a zoom level of one or more items are changed automatically. Also, in some examples, based on an assessment of the user's state and/or changes therein as indicated by the measured biometric, neurological and/or physiological data, a user interface of the device is automatically changed to highlight information (e.g., contextual information, links, etc.) and/or additional activities based on the area of engagement as reflected in the user's state(s).

Based on more information about a user's current state, changes or trends in the current user state, and/or a user's state history (e.g., as reflected in a neurological and/or physiological profile), some example devices are changed to automatically highlight semantic and/or image elements. In some examples, less or more items (e.g. a different number of element(s) or group(s) of element(s)) are chosen based on a user's state. In some examples, device characteristics that reflect placement of menus to facilitate fluent processing are chosen based on a user's state or profile. An example profile may include a history of a user's neurological and/or physiological states over time. Such a profile may provide a basis for assessing a user's current mental state relative to a user's baseline mental state. In such examples, the profile includes user preferences (e.g., affirmations—i.e. stated preferences—and/or observed preferences). Intra-state variations (e.g., a change insufficient to represent a change from a first state to a second state but presenting a trend toward such a state change) are monitored in some examples. Such intra-state change detections enable some example devices to adjust one or more characteristics to maintain a user in the current state or to push the user into a different state.

In addition to adapting or modifying a user device in accordance with user specific state(s) and/or profiles, examples disclosed herein identify and maintain affinity group profile(s) of physiological and/or neurological state preference(s) (e.g., articulated and/or observed), demographic preference(s) and/or baseline(s). The example group profile(s) may reflect affinity group(s) and/or neurological and/or physiological states and/or signatures across one or more populations (as observed and/or derived based on statistical techniques such as correlations). Some examples analyze groups to find signature correlates for a user or group and/or use advanced clustering algorithms to identify one or more affinity groups based on neurological and/or physiological state(s) and/or signature(s).

Aggregated usage data of an individual and/or group(s) of individuals are employed in some examples to identify patterns of states and/or to correlate patterns of user device attributes or characteristics. In some examples, test data from individual and/or group assessments (which may be either device specific and/or device independent), are completed to compile or otherwise develop a repository of user and/or group states and preferences. In some examples, neurological and/or physiological assessments of effectiveness of a user device characteristic are calculated and/or extracted by, for example, spectral analysis of neurological and/or physiological responses, coherence characteristics, inter-frequency coupling mechanisms, Bayesian inference, granger causality methods and/or other suitable analysis techniques. Such effectiveness assessments may be maintained in a repository or database and/or implemented on a device/interface for in-use assessments (e.g., real time assessment of the effectiveness of a device characteristic while a user is concurrently operating and/or interacting with the device).

In some examples, a group exhibits a significantly correlated device characteristic parsing and/or exploration pattern that may be leveraged to adapt a layout of information on the device to suit that group's behavior. In some examples, the presence or absence of complex background imagery is selected and/or modified while presenting foreground (e.g., semantic) information based on a group and/or individual profile.

In some examples, the user's information and the information of a group to which the user belongs are combined to provide a detailed assessment of the user's current state and/or a baseline assessment of the user's and/or users' state(s).

Examples disclosed herein evaluate neurological and/or physiological measurements representative of a current user state such as, for example, alertness, engagement and/or attention and adapt one or more aspects of a user device based on the measurement(s) and/or the user state. Examples disclosed herein are applicable to any type of user device including, for example, smart phone(s), mobile device(s), tablet(s), computer(s) and/or other machine(s). Some examples employ sensors such as, for example, cameras, detectors and/or monitors to collect one or more measurements such as pupillary dilation, body temperature, typing speed, grip strength, EEG measurements, eye movements, GSR data and/or other neurological, physiological and/or biometric data. In some such examples, if a user is identified as tired, drowsy, or otherwise not alert, an operating system, a browser, an application, a computer program and/or a user interface is automatically modified such that, for example, there is a change in display font sizes, a change in hues, a change in screen contrast and/or brightness, a change in volume, a change in content, a blocking of pop-up windows, etc. A change in any of these examples may be an increase or a decrease. If a user is very attentive, some example devices are modified to present more detail. A variety of device adjustments may be made based on user state, as detailed herein.

According to some examples, efforts are made to provide improved interfaces, applications and/or computer programs. Thus, for example, user interfaces, operating systems, browsers, application programs, machine interfaces, vehicle dashboards, etc., are dynamically and/or adaptively modified based on user neurological and/or physiological state information.

According to some examples, neuro-response data of a monitored user is analyzed to determine user state information. Neuro-response measurements such as, for example, central nervous system measurements, autonomic nervous system measurement and/or effector measurements may be used to evaluate a user as the user interacts with or otherwise operates a user device. Some examples of central nervous system measurement mechanisms that are employed in some examples detailed herein include functional magnetic resonance imaging (fMRI), EEG, MEG and optical imaging. Optical imaging may be used to measure the absorption or scattering of light related to concentration of chemicals in the brain or neurons associated with neuronal firing. MEG measures magnetic fields produced by electrical activity in the brain. fMRI measures blood oxygenation in the brain that correlates with increased neural activity.

EEG measures electrical activity resulting from thousands of simultaneous neural processes associated with different portions of the brain. EEG also measures electrical activity associated with post synaptic currents occurring in the milliseconds range. Subcranial EEG can measure electrical activity with high accuracy. Although bone and dermal layers of a human head tend to weaken transmission of a wide range of frequencies, surface EEG provides a wealth of useful electrophysiological information. In addition, portable EEG with dry electrodes also provides a large amount of useful neuro-response information.

EEG data can be classified in various bands. Brainwave frequencies include delta, theta, alpha, beta, and gamma frequency ranges. Delta waves are classified as those less than 4 Hz and are prominent during deep sleep. Theta waves have frequencies between 3.5 to 7.5 Hz and are associated with memories, attention, emotions, and sensations. Theta waves are typically prominent during states of internal focus. Alpha frequencies reside between 7.5 and 13 Hz and typically peak around 10 Hz. Alpha waves are prominent during states of relaxation. Beta waves have a frequency range between 14 and 30 Hz. Beta waves are prominent during states of motor control, long range synchronization between brain areas, analytical problem solving, judgment, and decision making. Gamma waves occur between 30 and 60 Hz and are involved in binding of different populations of neurons together into a network for the purpose of carrying out a certain cognitive or motor function, as well as in attention and memory. Because the skull and dermal layers attenuate waves in this frequency range, brain waves above 75-80 Hz may be difficult to detect. Nonetheless, in some of the disclosed examples, high gamma band (kappa-band: above 60 Hz) measurements are analyzed, in addition to theta, alpha, beta, and low gamma band measurements to determine a user's state (such as, for example, attention, emotional engagement and memory). In some examples, high gamma waves (kappa-band) above 80 Hz (detectable with sub-cranial EEG and/or magnetoencephalography) are used in inverse model-based enhancement of the frequency responses to user interaction with the user device. Also, in some examples, user and task specific signature sub-bands (i.e., a subset of the frequencies in a particular band) in the theta, alpha, beta, gamma and kappa bands are identified to estimate a user's state. Particular sub-bands within each frequency range have particular prominence during certain activities. In some examples, multiple sub-bands within the different bands are selected while remaining frequencies are band pass filtered. In some examples, multiple sub-band responses are enhanced, while the remaining frequency responses may be attenuated.

Autonomic nervous system measurement mechanisms that are employed in some examples disclosed herein include electrocardiograms (EKG) and pupillary dilation, etc. Effector measurement mechanisms that are employed in some examples disclosed herein include electrooculography (EOG), eye tracking, facial emotion encoding, reaction time, etc.

According to some examples, neuro-response data is generated from collected neurological, biometric and/or physiological data using a data analyzer that analyzes trends, patterns and/or relationships of data within a particular modality (e.g., EEG data) and/or between two or more modalities (e.g., EEG data and eye tracking data). Thus, the analyzer provides an assessment of intra-modality measurements and/or cross-modality measurements.

With respect to intra-modality measurement enhancements, in some examples, brain activity is measured to determine regions of activity and to determine interactions and/or types of interactions between various brain regions. Interactions between brain regions support orchestrated and organized behavior. Attention, emotion, memory, and other abilities are not based on one part of the brain but instead rely on network interactions between brain regions. In addition, different frequency bands used for multi-regional communication may be indicative of a user's state (e.g., a level of alertness, attentiveness and/or engagement). Thus, data collection using an individual collection modality such as, for example, EEG is enhanced by collecting data representing neural region communication pathways (e.g., between different brain regions). Such data may be used to draw reliable conclusions on user state (e.g., engagement level, alertness level, etc.) and, thus, to provide the bases for modifying one or more user characteristics of a computing device (e.g. a computer, a mobile phone, a tablet, an MP3 player, etc.). For example, if a user's EEG data shows high theta band activity at the same time as high gamma band activity, both of which are indicative of memory activity, an estimation may be made that the user's state is one of alertness, attentiveness and engaged. In response, a user device may be modified to provide more information to the user and/or to present content to a user at an accelerated rate.

With respect to cross-modality measurement enhancements, in some examples, multiple modalities to measure biometric, neurological and/or physiological data are used including, for example, EEG, GSR, EKG, pupillary dilation, EOG, eye tracking, facial emotion encoding, reaction time and/or other suitable biometric, neurological and/or physiological data. Thus, data collected from two or more data collection modalities may be combined and/or analyzed together to draw reliable conclusions on user states (e.g., engagement level, attention level, etc.). For example, activity in some modalities occur in sequence, simultaneously and/or in some relation with activity in other modalities. Thus, information from one modality may be used to enhance or corroborate data from another modality. For example, an EEG response will often occur hundreds of milliseconds before a facial emotion measurement changes. Thus, a facial emotion encoding measurement may be used to enhance the valence of an EEG emotional engagement measure. Also, in some examples EOG and eye tracking is enhanced by measuring the presence of lambda waves (a neurophysiological index of saccade effectiveness) in the EEG data in the occipital and extra striate regions of the brain, triggered by the slope of saccade-onset to estimate the significance of the EOG and eye tracking measures. In some examples, specific EEG patterns (i.e., signatures) of activity such as slow potential shifts and/or measures of coherence in time-frequency responses at the Frontal Eye Field (FEF) regions of the brain that preceded saccade-onset are measured to enhance the effectiveness of the saccadic activity data. Some such cross modality analyses employ a synthesis and/or analytical blending of central nervous system, autonomic nervous system and/or effector signatures. Data synthesis and/or analysis such as, for example, time and/or phase shifting, correlating and/or validating of intra-modal determinations with data collected from other data collection modalities allow for the generation of a composite output characterizing the significance of various data responses and, thus, the modification of one or more user characteristics of a computing device (e.g. a computer, a mobile phone, a tablet, an MP3 player, etc.) based on such a composite output.

According to some examples, actual expressed responses (e.g., survey data) and/or actions for one or more users or groups of users may be integrated with biometric, neurological and/or physiological data and stored in a database or repository in connection with one or more of a stimulus material, a user interface, an interface characteristic and/or an operating characteristic of a computing device.

Example method(s) of modifying op operating a user device disclosed herein include collecting at least one of biometric, neurological and/or physiological data of a user interacting with the user device. Such example method(s) also include identifying a current user state based on the at least one of the biometric, neurological and/or the physiological data, and modifying a characteristic of the user device based on the current user state and a desired user state.

Some example method(s) also include dynamically modifying a characteristic in real time or near real time to match, impede or drive changes in a user state, maintenance of a user state and/or changes between user states.

In some example(s), a user state is at least one of alert, attentive, engaged, disengaged, drowsy, distracted, confused, asleep or nonresponsive.

Some example method(s) also include modifying a characteristic of a user interface such as an automatic teller machine interface, a checkout display, a mobile phone display, a computer display, an airport kiosk, a home appliance display, a vending machine display, a tablet display, a portable music player display, a phone display and/or a vehicle dashboard.

Examples of modifying a characteristic of a user interface include changing a font size, changing a hue, changing a screen brightness, changing a screen contrast, changing a volume, changing content, blocking a pop-up window, allowing a pop-up window, changing an amount of detail, changing a language, adding personalization and/or changing a size of an icon.

In some example(s), neurological data includes one or more of functional magnetic resonance imaging data, electroencephalography data, magnetoencephalography data or optical imaging data. In some example(s), physiological data includes one or more of eye tracking data, tactile sensing data, head movement data, electrocardiogram data and/or galvanic skin response data.

Some example method(s) activate an alert to modify a characteristic. This is particularly useful if the user device is heavy machinery such as an automobile or an airplane.

Some example method(s) also include re-identifying or re-evaluating a user state after modifying a characteristic of a user device to determine an effectiveness of the modification.

Some example method(s) also include collecting data with a sensor separate from but operatively connected with, coupled to, integrated in and/or carried by a user device such as, for example, a mobile device (e.g., a phone) while a user operates the user device. In some examples, the sensor is incorporated into a housing when it will be controlled by a user head. In some examples, the sensor is implemented by a headset.

In some example method(s), a current user state is a desired user state and modifying a characteristic includes modifying the characteristic to maintain the user in the current user state.

In some example(s), one or more of the neurological and/or physiological data is collected from each user of a group of users and the collected data is combined to generate composite data. In such example(s), the characteristic is modified based on the composite data for a user operating the user device who is not a member of the group. Thus, the examples provide for a modification of a characteristic of a user device when there is no real time, recent or other observation or monitoring of a user. Also, in some examples, composite data includes one or more of data related to type of content of the user device, time of day of operation of the user device and/or task performed with the user device.

Example system(s) to operate and/or adjust (or operate by adjusting a characteristic of) a user device disclosed herein include a sensor to collect at least one of biometric, neurological and/or physiological data of a user interacting with the user device. Such example system(s) also include an analyzer to identify a current user state based on the at least one of the biometric, neurological and/or physiological data, and a characteristic adjuster to modify a characteristic of the user device based on the current user state and a desired user state.

In some example system(s), a characteristic adjuster is to dynamically modify one or more characteristic(s) of a user device in real time or near real time to match changes in a user state.

In some example system(s), a characteristic adjuster is to modify a characteristic of a user interface such as a characteristic of an automatic teller machine interface, a checkout display, a mobile phone display, a computer display, an airport kiosk, a home appliance display, a vending machine display, a tablet display, a portable music player display, a phone display and/or a vehicle dashboard.

In some example system(s), a characteristic adjuster is to modify a characteristic of a user interface by at least one of changing a font size, changing a hue, changing a screen brightness, changing a screen contrast, changing a volume, changing content, blocking a pop-up window, allowing a pop-up window, changing an amount of detail, changing a language, adding personalization and/or changing a size of an icon.

Some example system(s) also include an alarm to trigger an alert signal (e.g., a sound, a light, etc.) based on a user state.

In some example system(s), an analyzer is to re-identify a user state after a characteristic adjuster modifies a characteristic to determine an effectiveness of the modification.

In some example system(s), a sensor is coupled to, integrated in and/or carried by a mobile device (e.g., a phone) to measure neurological data while a user operates the mobile device.

In some example system(s), a current user state is a desired user state and a characteristic adjuster is to modify a characteristic to maintain the user in the current user state. Also, in some example(s), a current user state is not a desired state and a characteristic adjuster is to modify a characteristic to change the user state.

Example machine readable medium disclosed herein stores instructions thereon which, when executed, cause a machine to at least collect at least one of biometric, neurological and/or physiological data of a user interacting with a user device. In addition the example instructions cause a machine to identify a current user state based on the at least one of the biometric, neurological and/or physiological data and to modify a characteristic of the user device based on the current user state and a desired user state.

Some example instructions cause a machine to dynamically modify a characteristic in real time or near real time to match changes in a user state.

Some example instructions cause a machine to modify a characteristic of a user interface such as an automatic teller machine interface, a checkout display, a mobile phone display, a computer display, an airport kiosk, a home appliance display, a vending machine display, a tablet display, a portable music player display, a phone display and/or a vehicle dashboard.

Some example instructions cause a machine to modify a characteristic of a user interface by at least one of changing a font size, changing a hue, changing a screen brightness, changing a screen contrast, changing a volume, changing content, blocking a pop-up window, allowing a pop-up window, changing an amount of detail, changing a language, adding personalization and/or changing a size of an icon.

Some example instructions further cause a machine to activate an alert based on a user state.

Some example instructions further cause a machine to re-identify a user state after modifying a characteristic to determine an effectiveness of the modification.

Some example instructions cause a machine to collect biometric, neurological and/or physiological data with a sensor coupled to, integrated in and/or carried by a mobile device (e.g., a phone) while a user operates the mobile device.

In some example, a current user state is a desired user state and the instructions further cause a machine to modify a characteristic to maintain the user in the current user state.

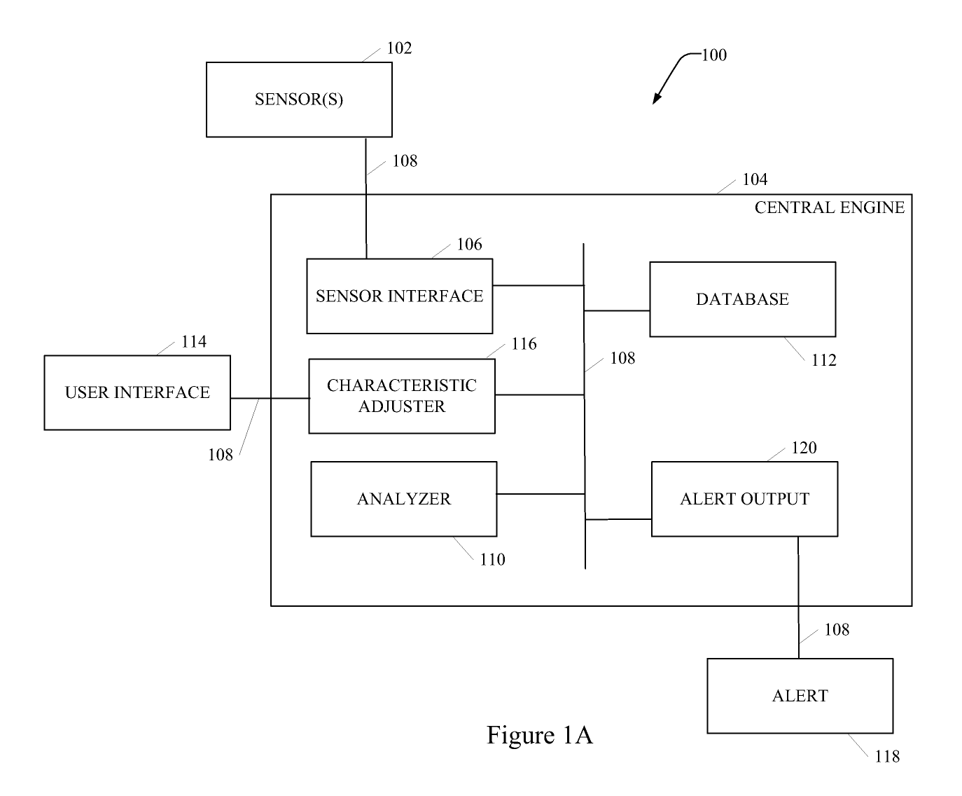

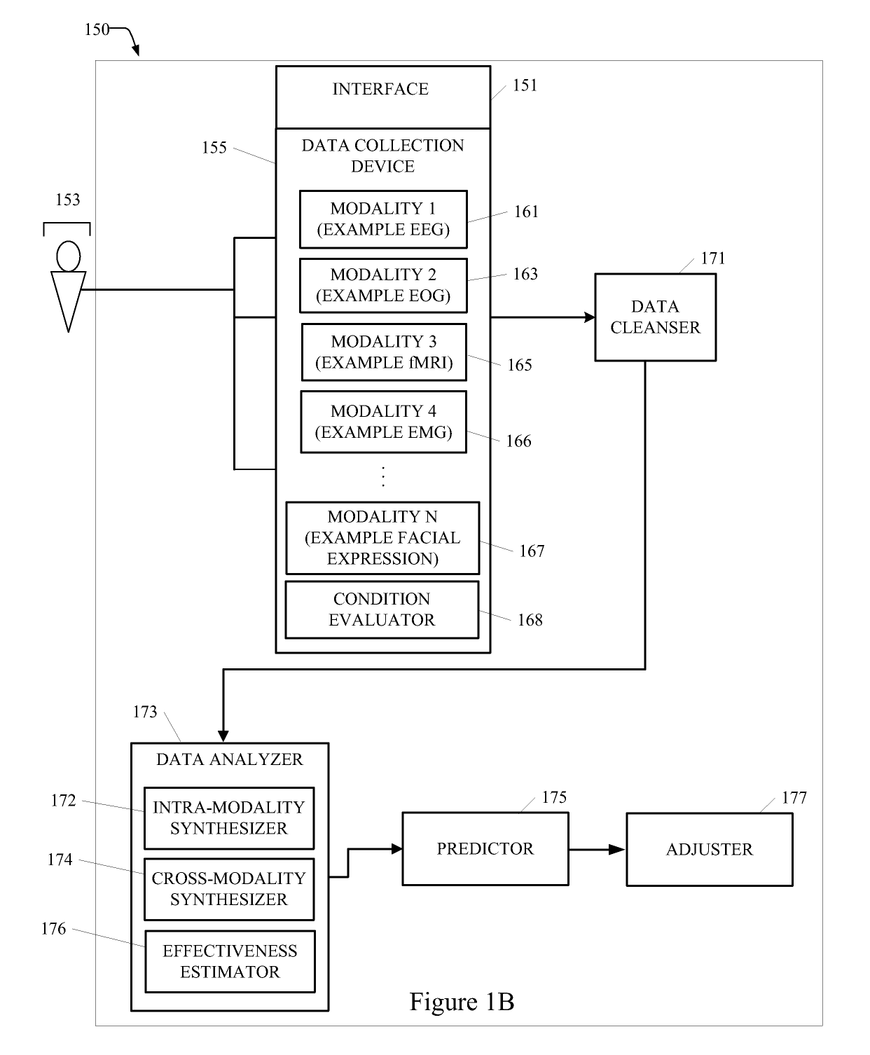

Turning to the figures, FIG. 1A illustrates an example system 100 that may be used to gather neurological, physiological and/or biometric data of a user operating a user device. The user device has a characteristic to be adjusted based on the user's state (as represented by collected data) and a desired state. The collected data of the illustrated example is analyzed to determine the user's current state (e.g., a user's emotions and conditions, attention level, alertness, engagement level, response ability, vigilance, and/or how observant the user currently is). The information about the user's current state(s) may be compared with one or more profiles to select a corresponding device characteristic (e.g., a user interface characteristic) to modify to adapt the device to the user's current neurological and/or physiological condition in real time, substantial real time and/or periodically. The example system 100 of FIG. 1A includes one or more sensor(s) 102. The sensor(s) 102 of the illustrated example gather one or more of user neurological data or user physiological data. The sensor(s) 102 may include, for example, one or more electrode(s), camera(s) and/or other sensor(s) to gather any type of data described herein (including, for example, functional magnetic resonance imaging data, electroencephalography data, magnetoencephalography data and/or optical imaging data). The sensor(s) 102 may gather data continuously, periodically or aperiodically.

The example system 100 of FIG. 1A includes a central engine 104 that includes a sensor interface 106 to communicate with the sensor(s) 102 over communication links 108. The communication links 108 may be any type of wired (e.g., a databus, a USB connection, etc.) or wireless communication mechanism (e.g., radio frequency, infrared, etc.) using any past, present or future communication protocol (e.g., Bluetooth, USB 2.0, etc.).

The example system 100 of FIG. 1A also includes an analyzer 110, which examines the data gathered by the sensor(s) 102 to determine a current user state. For example, if the analyzer 110 examines the data collected by the sensor(s) 102 and determines that the user has slow eye tracking, droopy eyelids, slow breathing and/or EEG data that shows increasing delta wave activity indicating sleepiness, the analyzer 110 of the instant examples, concludes that the user is in a state of low engagement and is not alert or attentive. The analyzer 110 of the instant example then identifies one or more characteristics of the device being operated by the user (e.g., an interface) that correlates and/or matches with moving a sleepy person into a more alert state such as, for example, a brighter screen, higher volume, audible alert, vibration or larger font size. In examples in which sleepiness is not being resisted (and may be promoted), the analyzer 110 may alternatively reduce screen brightness, reduce the volume and/or shut off the device. In other words, the analyzer 110 identifies one or more device characteristics appropriate for modification to provide a desired result based on the current user state. Characteristics amenable to modification may be catalogued or otherwise mapped to user states and stored in a database 112. The database 112 of the illustrated example records a history of a user's states to develop a user profile including, for example, a user baseline to facilitate identification and/or classification of the current user state.

The analyzer 110 of the illustrated example communicates the identified user state(s) and/or one or more characteristics corresponding to the current user's state(s) to a characteristic adjuster 116 via a communication link 108. The adjuster 116 then adjusts one or more characteristic(s) to match the user's current state(s), to attempt to maintain a user in the current state and/or to attempt to produce a desired change in the user's state(s). For example, the adjuster 116 may change an operating speed of the device (e.g., to conserve power) and/or may change one or more characteristic(s) of a program, application and/or user interface 114. Example user interfaces 114 include one or more of an automatic teller machine interface, a checkout display, a phone display, a computer display, an airport kiosk, a home appliance display, a vending machine display, a tablet display, a portable music player display and a vehicle dashboard.

The user interface 114, the sensor(s) 102 and/or the central engine 104 (and/or components thereof) of the illustrated example may be integrated in the controlled user device or distributed over two or more devices. For example, where the user interface 114 is a mobile phone interface, the sensor(s) 102 may be coupled to, integrated in and/or carried by the mobile phone, externally or internally, to measure the user's biometric, neurological and/or physiological data while the user operates the mobile phone (e.g., via the hands of the user, via a camera of the device, etc.). In such examples, the analyzer 110 and/or the database 112 are incorporated into the user device. In some examples, the analyzer 110 and the database 112 are located remotely from the user device.

The example adjuster 116 of FIG. 1A provides instructions to modify a characteristic of the user interface 114 based on the current user state(s) and/or desired user state. The user interface 114 may be modified in any way including, for example, by changing a font size, changing a hue, changing a screen brightness, changing a screen contrast, changing a volume, changing content, blocking a pop-up window, allowing a pop-up window, changing an amount of detail presented via the user interface, changing a language (e.g., from a second language to a native language), adding personalization, issuing an alert (e.g., a sound, a visible message, a vibration, etc.) and/or changing a size of an icon. As used in these examples, changing may be an increase or a decrease, depending on the desired result.

In the illustrated example, the central engine 104 continually operates to dynamically modify one or more characteristics of the user device (e.g., one or more aspects of the user interface 114) in real time or near real time to match changes in the user's state(s), to maintain a current user state and/or to change a current user state. Thus, the characteristics of the user device may be modified to track the user's state or to attempt to effect the user's state. In the illustrated example, the analyzer 110 continues to analyze the collected biometric, neurological and/or physiological data after the modification to determine an effectiveness of the modification in achieving the desired result (e.g., changing a user state, maintaining a user state, etc.). Further, the sensor(s) 102, the analyzer 110 and the adjuster 116 may cooperate to form a feedback loop. As a result, ineffective changes may result in further modifications until the analyzer 110 determines that a change was effective in achieving the desired result. For example, if a sleepy user is not awakened by a brighter screen, the adjuster 116 may instruct the user interface 114 to modify the volume to an increased level. Further, some adjustments may be temporary and, thus, removed or modified once the desired state change is achieved (e.g., the volume may be lowered).

In the illustrated example, a set of baseline states for a user are determined and stored in the database 112. The baseline states are useful because different people have different characteristics and behaviors. The baseline states assist the example system 100 and, in particular, the analyzer 110 in classifying a current user state and/or in determining when a user's state has or has not changed. (As noted above, either a change in state or no change in state may be an indication that further modification(s) to the device characteristic(s) are warranted. For example, a failure to change state in response to an adjustment may indicate that another adjustment should be affected.). In some examples, a normally calm person may have a period of heightened excitement and activity that could cause the analyzer 110 and/or the adjuster 116 to instruct the user interface 114 to include more detail. However, a normally active or fidgety person may not require any changes in the user interface 114 even though the same absolute data values as the normally calm person are measured. The baseline state information facilitates changes in the device (e.g., in the user interface 114) based on relative user state changes for a particular user.

The example system 100 of FIG. 1A also includes an alert 118 that is coupled to the central engine 104 via an alert output 120 and the communication links 108 (e.g., a bus). The alert 118 may be triggered based on a user state. For example, when the analyzer 110 determines that a user is in a drowsy state, an audio alarm may sound to grab the user's attention. In some examples, the system 100 may be incorporated into an automobile. When it is detected that the driver is drowsy, a loud noise may sound in the automobile to bring the driver to a heightened state of alert and increase the safety of the driving.

FIG. 1B illustrates an example user device 150 having a characteristic that may be adaptively modified based on the neurological and/or physiological state of a user. The example device 150 includes a user interface 151, which may be implemented by, for example, a display, a monitor, a screen and/or other device to display information to a user.

In the illustrated examples, a user 153 is monitored by one or more data collection devices 155. The data collection devices 155 may include any number or types of neuro-response measurement mechanisms such as, for example, neurological and neurophysiological measurement systems such as EEG, EOG, MEG, pupillary dilation, eye tracking, facial emotion encoding and/or reaction time devices, etc. In some examples, the data collection devices 155 collect neuro-response data such as central nervous system, autonomic nervous system and/or effector data. In some examples, the data collection devices 155 include components to gather EEG data 161, components to gather EOG data 163 and/or components to gather fMRI data 165. In some examples, only a single data collection device 155 is used. In other examples a plurality of collection devices 155 are used. Data collection is performed automatically in the illustrated example. That it, data collection is performed without a user's involvement other than engagement with the sensor(s) 102.

The data collection device(s) 155 of the illustrated example collect neuro-response data from multiple sources and/or modalities. Thus, the data collection device(s) 155 include a combination of devices to gather data from central nervous system sources (EEG), autonomic nervous system sources (EKG, pupillary dilation) and/or effector sources (EOG, eye tracking, facial emotion encoding, reaction time). In some examples, the data collected is digitally sampled and stored for later analysis. In some examples, the data collected is analyzed in real-time. According to some examples, the digital sampling rates are adaptively chosen based on the biometric, physiological, neurophysiological and/or neurological data being measured.

In the illustrated example, the data collection device 155 collects EEG measurements 161 made using scalp level electrodes, EOG measurements 163 made using shielded electrodes to track eye data, fMRI measurements 165 performed using a differential measurement system, EMG measurements 166 to measure facial muscular movement through shielded electrodes placed at specific locations on the face and a facial expression measurement 167 that includes a video analyzer.

In some examples, the data collection devices 155 are clock synchronized with the user interface 151. In some examples, the data collection devices 155 also include a condition evaluator 168 that provides auto triggers, alerts and/or status monitoring and/or visualization components that continuously or substantially continuously (e.g., at a high sampling rate) monitor the status of the subject, the data being collected and the data collection instruments. The condition evaluator 168 may also present visual alerts and/or automatically trigger remedial actions.

According to some examples, the user interface presentation system also includes a data cleanser device 171. The example data cleanser device 171 of the illustrated example filters the collected data to remove noise, artifacts, and/or other irrelevant data using any or all of fixed and/or adaptive filtering, weighted averaging, advanced component extraction (like PCA, ICA), vector and/or component separation methods, etc. The data cleanser 171 cleanses the data by removing both exogenous noise (where the source is outside the physiology of the subject, e.g. a phone ringing while a subject is viewing a video) and endogenous artifacts (where the source could be neurophysiological, e.g. muscle movements, eye blinks, etc.).

The artifact removal subsystem of the data cleanser 171 of the illustrated example, includes mechanisms to selectively isolate and review the response data and/or identify epochs with time domain and/or frequency domain attributes that correspond to artifacts such as line frequency, eye blinks, and/or muscle movements. The artifact removal subsystem then cleanses the artifacts by either omitting these epochs, or by replacing this epoch data with an estimate based on the other clean data (for example, an EEG nearest neighbor weighted averaging approach).

The data cleanser device 171 of the illustrated example may be implemented using hardware, firmware, and/or software. It should be noted that although a data cleanser device 171 is shown located after a data collection device 155, the data cleanser device 171 like other components may have a different location and/or functionality based on system implementation. For example, some systems may not use any automated data cleanser device while in other systems, data cleanser devices may be integrated into individual data collection devices.

In the illustrated example, the user device 150 includes a data analyzer 173. The example data analyzer 173 analyzes the neurological and/or physiological data collected by the data collection device 155 to determine a user's current state(s). In some examples, the data analyzer 173 generates biometric, neurological and/or physiological signatures from the collected data using time domain analyses and/or frequency domain analyses. Such analyses may use parameters that are common across individuals and/or parameters that are unique to each individual. The analyses may utilize statistical parameter extraction and/or fuzzy logic to determine a user state from the time and/or frequency components. In some examples, statistical parameters used in the user state determination include evaluations of skew, peaks, first and second moments and/or distribution of the collected data.

In some examples, the data analyzer 173 includes an intra-modality response synthesizer 172 and a cross-modality response synthesizer 174. The intra-modality response synthesizer 172 analyzes intra-modality data as disclosed above. The cross-modality response synthesizer 174 analyzer data from two or more modalities as disclosed above.

In the illustrated example, the data analyzer 173 also includes an effectiveness estimator 176 that analyzes the data to determine an effectiveness of modifying a user device characteristic in producing a desired result, such as changing or maintaining a desired user state. For example, biometric, neurological and/or physiological data is collected subsequent to a modification in a user device and analyzed to determine if a user state has changed or been maintained in accordance with the desired result.

In some examples, the collected data is analyzed by a predictor 175, which generates patterns, responses, and/or predictions. For example, in the illustrated example, the predictor 175 compares biometric, neurological and/or physiological data (e.g., data reflecting patterns and expressions for the current user and/or for a plurality of users) to predict a user's current state and/or an impending state. In some examples, patterns and expressions are combined with survey, demographic and/or stated and/or observed preference data. An operating condition (e.g., a user interface characteristic) of the user device 150 may be changed based on the current user state and/or the prediction(s) of the predictor 150.

The example system of FIG. 1B also includes a characteristic adjuster 177 that adjusts a characteristic of a user device (e.g., a characteristic of a user interface) based on the user's state. The adjuster 177 operates in a manner similar to the adjuster 116 of FIG. 1A.

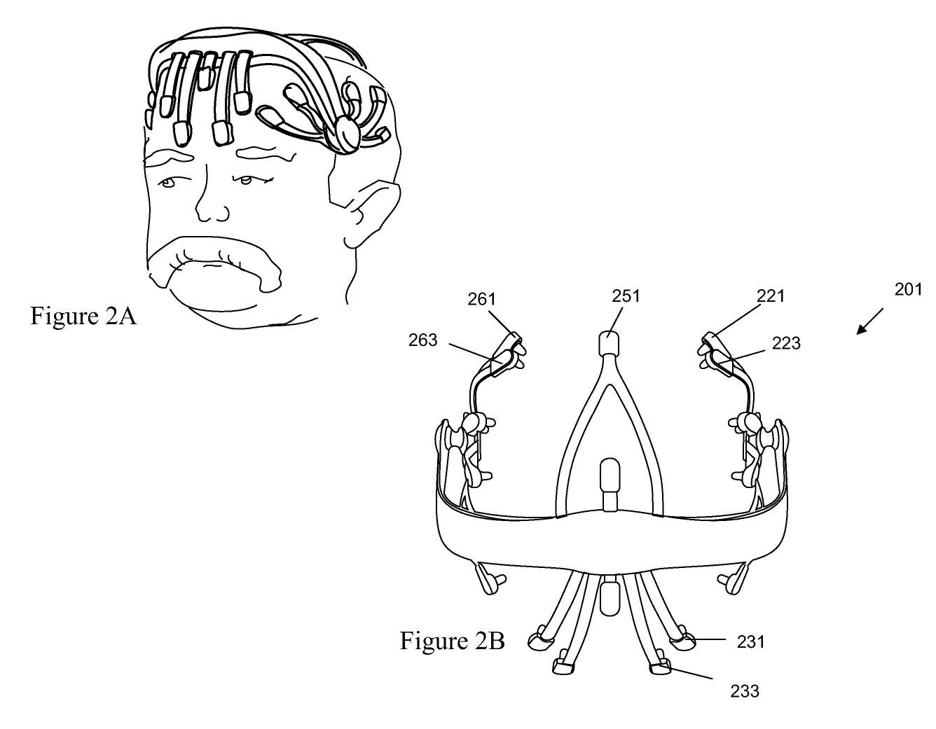

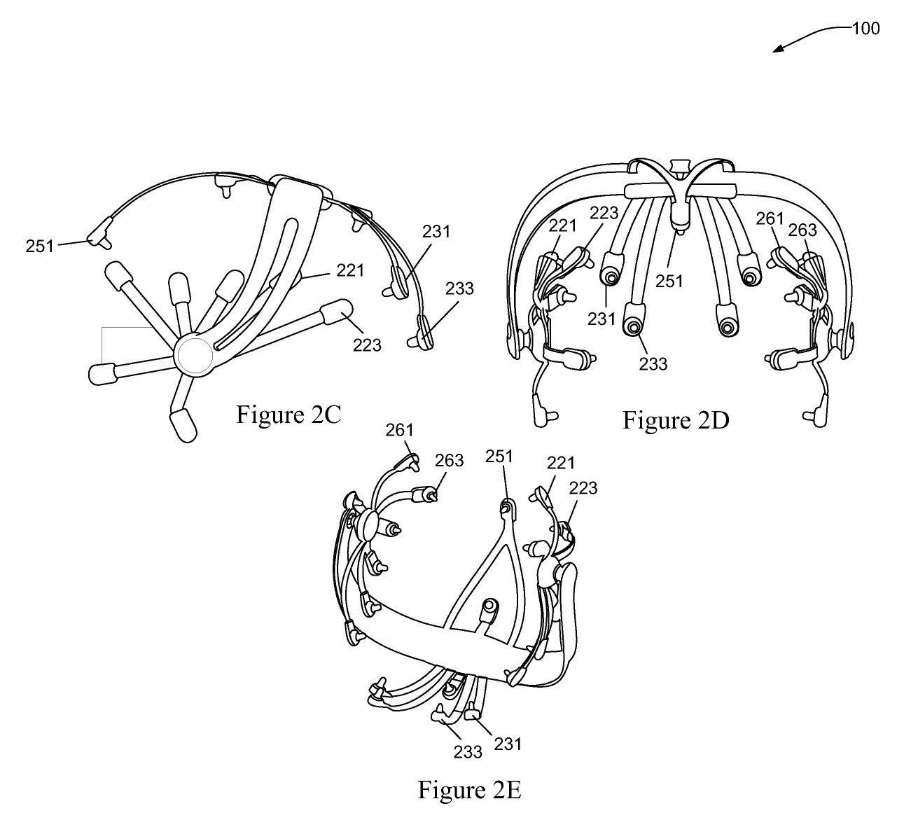

FIGS. 2A-2E illustrate an example data collector 201, which in this example, collects neurological data. FIG. 2A shows a perspective view of the data collector 201 including multiple dry electrodes. The illustrated example data collector 201 is a headset having point or teeth, dry electrodes to contact the scalp through human hair without the use of electro-conductive gels. In some examples, the signal collected by each electrode is individually amplified and isolated to enhance shielding and routability. In some examples, each electrode has an associated amplifier implemented using a flexible printed circuit. Signals may be routed to a controller/processor for immediate transmission to a data analyzer or stored for later analysis. A controller/processor may be used to synchronize data with a user device. The data collector 201 may also have receivers for receiving clock signals and processing neurological signals. The data collector 201 may also have transmitters for transmitting clock signals and sending data to a remote entity such as a data analyzer.

FIGS. 2B-2E illustrate top, side, rear, and perspective views of the data collector 201. The example data collector 201 includes multiple dry electrodes including right side electrodes 261 and 263, left side electrodes 221 and 223, front electrodes 231 and 233, and rear electrode 251. The specific electrode arrangement may be different in other examples. In the illustrated example, the placing of electrodes on the temporal region of the head is avoided to prevent collection of signals generated based on muscle contractions. Avoiding contact with the temporal region also enhances comfort during sustained wear.

In some examples, forces applied by the electrodes 221 and 223 counterbalance forces applied by the electrodes 261 and 263, and forces applied by the electrodes 231 and 233 counterbalance forces applied by electrode 251. Also, in some examples, the EEG dry electrodes detect neurological activity with little or no interference from human hair and without use of any electrically conductive gels. Also, in some examples, the data collector 201 also includes EOG sensors such as sensors used to detect eye movements.

In some examples, data acquisition using the electrodes 221, 223, 231, 233, 251, 261, and 263 is synchronized with changes in a user device such as, for example, changes in a user interface. Data acquisition can be synchronized with the changes in the user device by using a shared clock signal. The shared clock signal may originate from the user device, a headset, a cell tower, a satellite, etc. The data collection mechanism 201 also includes a transmitter and/or receiver to send collected data to a data analysis system and to receive clock signals as needed. In some examples, a transceiver transmits all collected data such as biometric data, neurological data, physiological data, user state and sensor data to a data analyzer. In other examples, a transceiver transmits only select data provided by a filter.

In some examples, the transceiver may be coupled to a computer system that transmits data over a wide area network to a data analyzer. In other examples, the transceiver directly sends data to a local data analyzer. Other components such as fMRI and MEG that are not yet portable but may become portable at some future time may also be integrated into a headset.

In some examples, the data collector 201 includes, for example, a battery to power components such as amplifiers and transceivers. Similarly, the transceiver may include an antenna. Also, in some examples, some of the components are excluded. For example, filters or storage may be excluded.

While example manners of implementing the example system to modify a user device of FIG. 1A, the example user device of FIG. 1B and the example data collection apparatus of FIGS. 2A-E have been disclosed herein and illustrated in the respective figures, one or more of the elements, processes and/or devices illustrated in FIGS. 1A, 1B and 2A-E may be combined, divided, re-arranged, omitted, eliminated and/or implemented in any other way. Further, the example central engine 104, the example sensor(s) 102, the example sensor interface 106, the example analyzer 110, the example, database 112, the example user interface 114, the example adjuster 116, the example alert 118, the example alert output 120, the example user interface 151, the example data collection device(s) 155, the example data cleanser 171, the example data analyzer 173, the example predictor 175, the example adjuster 177 and/or, more generally, the example system 100, the example user device 150 and/or the example data collector 201 may be implemented by hardware, software, firmware and/or any combination of hardware, software and/or firmware. Thus, for example, any of the example central engine 104, the example sensor(s) 102, the example sensor interface 106, the example analyzer 110, the example, database 112, the example user interface 114, the example adjuster 116, the example alert 118, the example alert output 120, the example user interface 151, the example data collection device(s) 155, the example data cleanser 171, the example data analyzer 173, the example predictor 175, the example adjuster 177 and/or, more generally, the example system 100, the example user device 150 and/or the example data collector 201 could be implemented by one or more circuit(s), programmable processor(s), application specific integrated circuit(s) (ASIC(s)), programmable logic device(s) (PLD(s)) and/or field programmable logic device(s) (FPLD(s)), etc. When any of the appended apparatus or system claims are read to cover a purely software and/or firmware implementation, at least one of the example central engine 104, the example sensor(s) 102, the example sensor interface 106, the example analyzer 110, the example, database 112, the example user interface 114, the example adjuster 116, the example alert 118, the example alert output 120, the example user interface 151, the example data collection device(s) 155, the example data cleanser 171, the example data analyzer 173, the example predictor 175, the example adjuster 177 are hereby expressly defined to include a tangible computer readable medium such as a memory, DVD, CD, etc. storing the software and/or firmware. Further still, the example system 100, the example user device 150 and/or the example data collector 201 may include one or more elements, processes and/or devices in addition to, or instead of, those illustrated in FIGS. 1A, 1B and/or 2A-E, and/or may include more than one of any or all of the illustrated elements, processes and devices.

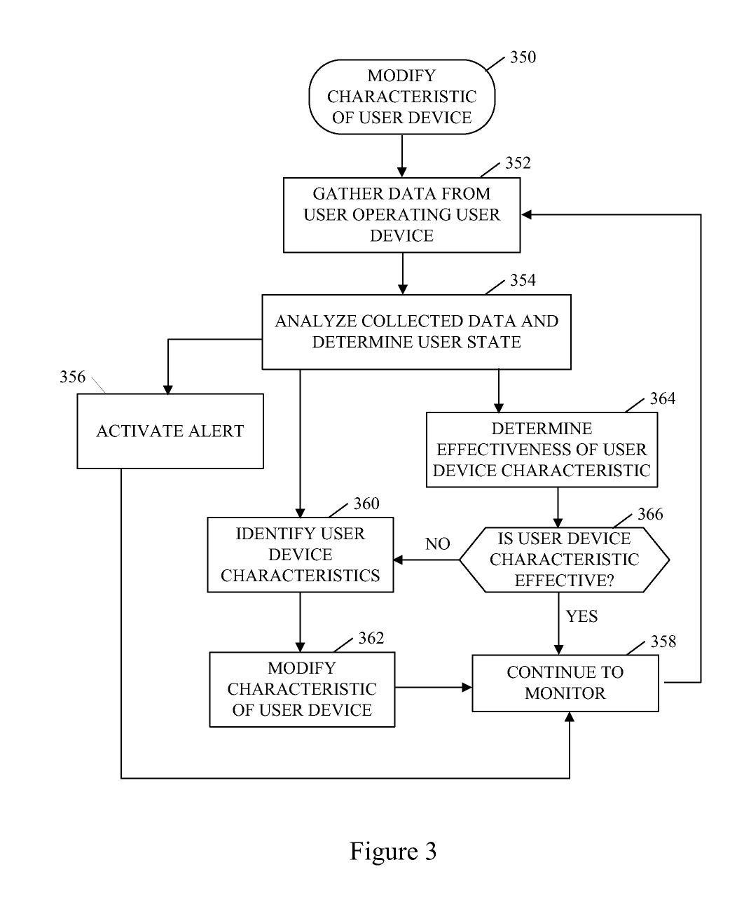

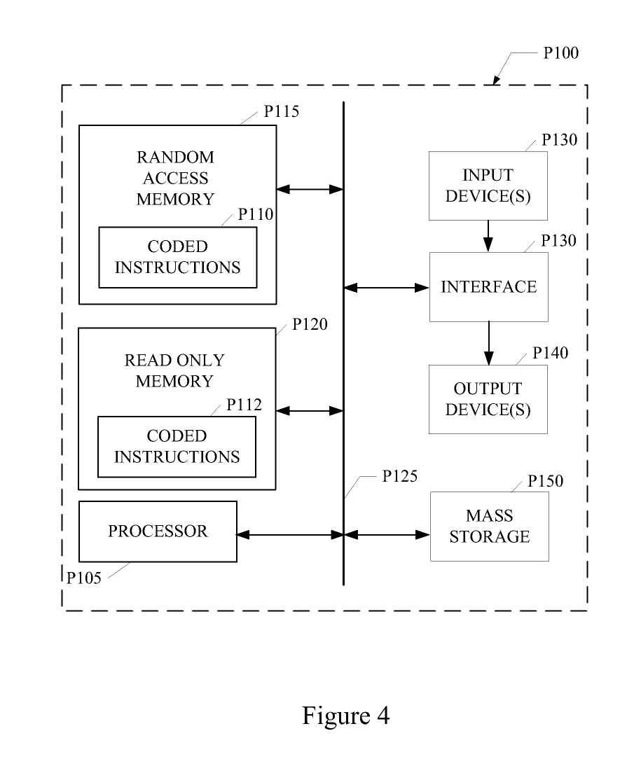

FIG. 3 is a flowchart representative of example machine readable instructions that may be executed to implement the example system 100, the example central engine 104, the example sensor(s) 102, the example sensor interface 106, the example analyzer 110, the example, database 112, the example user interface 114, the example adjuster 116, the example alert 118, the example alert output 120, the example user device 150, the example user interface 151, the example data collection device(s) 155, the example data cleanser 171, the example data analyzer 173, the example predictor 175, the example adjuster 177, the example data collector 201 and/or other components of FIGS. 1A, 1B and 2A-2E. In the examples of FIG. 3, the machine readable instructions include a program for execution by a processor such as the processor P105 shown in the example computer P100 discussed below in connection with FIG. 4. The program may be embodied in software stored on a tangible computer readable medium such as a CD-ROM, a floppy disk, a hard drive, a digital versatile disk (DVD), or a memory associated with the processor P105, but the entire program and/or parts thereof could alternatively be executed by a device other than the processor P105 and/or embodied in firmware or dedicated hardware. Further, although the example program is described with reference to the flowchart illustrated in FIG. 3, many other methods of implementing the example system 100, the example central engine 104, the example sensor(s) 102, the example sensor interface 106, the example analyzer 110, the example, database 112, the example user interface 114, the example adjuster 116, the example alert 118, the example alert output 120, the example user device 150, the example user interface 151, the example data collection device(s) 155, the example data cleanser 171, the example data analyzer 173, the example predictor 175, the example adjuster 177, the example data collector 201 and other components of FIGS. 1A, 1B and 2A-2E may alternatively be used. For example, the order of execution of the blocks may be changed, and/or some of the blocks described may be changed, eliminated, or combined.

As mentioned above, the example processes of FIG. 3 may be implemented using coded instructions (e.g., computer readable instructions) stored on a tangible computer readable medium such as a hard disk drive, a flash memory, a read-only memory (ROM), a compact disk (CD), a digital versatile disk (DVD), a cache, a random-access memory (RAM) and/or any other storage media in which information is stored for any duration (e.g., for extended time periods, permanently, brief instances, for temporarily buffering, and/or for caching of the information). As used herein, the term tangible computer readable medium is expressly defined to include any type of computer readable storage and to exclude propagating signals. Additionally or alternatively, the example processes of FIG. 3 may be implemented using coded instructions (e.g., computer readable instructions) stored on a non-transitory computer readable medium such as a hard disk drive, a flash memory, a read-only memory, a compact disk, a digital versatile disk, a cache, a random-access memory and/or any other storage media in which information is stored for any duration (e.g., for extended time periods, permanently, brief instances, for temporarily buffering, and/or for caching of the information). As used herein, the term non-transitory computer readable medium is expressly defined to include any type of computer readable medium and to exclude propagating signals.

FIG. 3 illustrates another example process to modify or adjust an operating characteristic of a user device (block 350). The example method 350 includes gathering biometric, neurological and/or physiological data from a user operating the user device (block 352) via, for example, the sensor(s) 102, 201 described above. The example method 350 also includes analyzing the collected data to determine a user state (block 354). The biometric, neurological and/or physiological data may be analyzed (block 354) using, for example, the analyzer 110 or other devices described above.

Upon analyzing the biometric, neurological and/or physiological data and determining a current user state (block 354), the example process 350 may proceed with one or more actions corresponding to the current user state and/or a desired result. For example, the example process 350 may activate an alert (block 356). For example, as detailed above, an audible alert may sound to awaken a sleepy user. After an alert is activated (block 356), the example process 350 may continue to monitor user state data (block 358).

Additionally or alternatively, when the biometric, neurological and/or physiological data is analyzed and the current user state is determined (block 354), the example process 350 may identify one or more user device characteristics (block 360) that correlate with the determined user state, a tendency to maintain the current user state and/or a tendency to change a current user state toward a desired user state. The desired user state may be predicted by the user, by an advertiser, by an application program, by the device manufacturer and/or by any other entity and may be tied to environmental factors such as time of day, geographic location (e.g., as measured by a GPS device, etc.). The example process 350 may correlate the current user state with one or more device characteristics using for example, the analyzer 110, the database 112, the adjuster 116, the analyzer 173, the predictor 175 and/or the adjuster 177.

The example process 350 of the illustrated example modifies a characteristic of the user device (block 362) (e.g., the interface 114 of FIG. 1A and/or the user interface 151 of FIG. 1B) in accordance with the identified device characteristics (block 360). The device may be modified in accordance with one or more of the modifications described above. After the device is modified (block 362), the example process 350 may continue to monitor biometric, neurological and/or physiological data (block 358).

Additionally or alternatively, when the collected data is analyzed and the user state is determined (block 354), the example process 350 may determine the effectiveness of a user device characteristic or a previous adjustment to a user device characteristic (block 364). The effectiveness may be determined using, for example, a feedback loop comprising the analyzer 110, the database 112 and/or the adjuster 116, or comprising the analyzer 173, the predictor 175 and/or the adjuster 177, as described above. For example, if the gathered biometric, neurological and/or physiological data (block 352) is analyzed (block 354) and indicates that user state has not changed in a desired way after a modification of the user device (bock 362), the process 350 may determine that the adjustment to the user device characteristic was not effective (blocks 364, 366). However, if the gathered biometric, neurological and/or physiological data (block 352) is analyzed (block 354) and indicates that the user has behaved in a desired way after the modification of the characteristic of the user device (block 362), the process 350 may determine that the adjustment to user device characteristic was effective (blocks 364, 366).

If the user device characteristic adjustment is not effective (block 366), then the process returns to block 360 where one or more additional adjustments and/or device characteristics are identified for adjustment to attempt to effect the desired result in the user state. If a further adjustment to a user device characteristic is effective (block 366), the example process 350 continues to monitor the user (block 358).

FIG. 4 is a block diagram of an example processing platform P100 capable of executing the instructions of FIG. 3 to implement the example system 100, the example central engine 104, the example sensor(s) 102, the example sensor interface 106, the example analyzer 110, the example, database 112, the example user interface 114, the example user interface interface 116, the example alert 118, the example alert output 120, the example system 150, the example presentation device 151, the example data collection device(s) 155, the example data cleanser 171, the example data analyzer 173, the example predictor 175, the example adjuster 177, the example data collector 201. The processor platform P100 can be part of, for example, any user device such as a mobile device, a telephone, a cell phone, a tablet, an MP3 player, a game player, a server, a personal computer, or any other type of computing device.

The processor platform P100 of the instant example includes a processor P105. For example, the processor P105 can be implemented by one or more Intel® microprocessors. Of course, other processors from other families are also appropriate.

The processor P105 is in communication with a main memory including a volatile memory P115 and a non-volatile memory P120 via a bus P125. The volatile memory P115 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory P120 may be implemented by flash memory and/or any other desired type of memory device. Access to the main memory P115, P120 is typically controlled by a memory controller.

The processor platform P100 also includes an interface circuit P130. The interface circuit P130 may be implemented by any type of past, present or future interface standard, such as an Ethernet interface, a universal serial bus (USB), and/or a PCI express interface.

One or more input devices P135 are connected to the interface circuit P130. The input device(s) P135 permit a user to enter data and commands into the processor P105. The input device(s) can be implemented by, for example, a keyboard, a mouse, a touchscreen, a track-pad, a trackball, isopoint and/or a voice recognition system.

One or more output devices P140 are also connected to the interface circuit P130. The output devices P140 can be implemented, for example, by display devices (e.g., a liquid crystal display, and/or a cathode ray tube display (CRT)). The interface circuit P130, thus, typically includes a graphics driver card.

The interface circuit P130 also includes a communication device, such as a modem or network interface card to facilitate exchange of data with external computers via a network (e.g., an Ethernet connection, a digital subscriber line (DSL), a telephone line, coaxial cable, a cellular telephone system, etc.).

The processor platform P100 also includes one or more mass storage devices P150 for storing software and data. Examples of such mass storage devices P150 include floppy disk drives, hard drive disks, compact disk drives and digital versatile disk (DVD) drives.

The coded instructions of FIG. 3 may be stored in the mass storage device P150, in the volatile memory P110, in the non-volatile memory P112, and/or on a removable storage medium such as a CD or DVD.

Although certain example methods, apparatus and articles of manufacture have been described herein, the scope of coverage of this patent is not limited thereto. On the contrary, this patent covers all methods, apparatus and articles of manufacture fairly falling within the scope of the claims of this patent.

What is claimed is:

1. A method of operating a user device, the method comprising:
collecting at least one of neurological data or physiological data of a user interacting with the user device;
identifying a current user state based on the at least one of the neurological data or the physiological data; and
modifying a characteristic of the user device based on the current user state and a desired user state.

2. The method as defined in claim 1, wherein modifying the characteristic of the user device comprises dynamically modifying the characteristic in real time or near real time to match changes in the user state.

3. The method as defined in claim 1, wherein the user state comprises at least one of alert, attentive, engaged, disengaged, drowsy, distracted, confused, asleep or nonresponsive.

4. The method of as defined in claim 1, wherein modifying the characteristic comprises modifying a characteristic of a user interface, and the user interface comprises at least one of an automatic teller machine interface, a checkout display, a mobile phone display, a computer display, an airport kiosk, a home appliance display, a vending machine display, a tablet display, a portable music player display, a phone display or a vehicle dashboard.

5. The method as defined in claim 1, wherein modifying the characteristic comprises modifying a characteristic of a user interface by at least one of changing a font size, changing a hue, changing a screen brightness, changing a screen contrast, changing a volume, changing content, blocking a pop-up window, allowing a pop-up window, changing an amount of detail, changing a language, adding personalization or changing a size of an icon.

6. The method as defined in claim 1, wherein the neurological data includes one or more of functional magnetic resonance imaging data, electroencephalography data, magnetoencephalography data or optical imaging data.

7. The method as defined in claim 1 wherein modifying the characteristic comprises activating an alert.

8. The method as defined in claim 1 further comprising re-identifying the user state after modifying the characteristic to determine an effectiveness of the modification.

9. The method as defined in claim 1, wherein collecting the neurological data or the physiological data comprises collecting the data with a sensor associated with the user device while the user operates the user device.

10. The method as defined in claim 1, wherein the current user state is the desired user state and modifying the characteristic comprises modifying the characteristic to maintain the user in the current user state.

11. The method as defined in claim 1, wherein the physiological data includes one or more of eye tracking data, tactile sensing data, head movement data, electrocardiogram data or galvanic skin response data.

12. The method as defined in claim 1, wherein one or more of the neurological or physiological data is collected from each user of a group of users and the collected data is combined to generate composite data and the characteristic is modified based on the composite data for a user operating the user device who is not a member of the group.

13. The method as defined in claim 12, wherein the composite data further includes one or more of data related to type of content of the user device, time of day of operation of the user device or task performed with the user device.

14. A system to operate a user device, the system comprising:
a sensor to collect at least one of neurological data or physiological data of a user interacting with the user device;
an analyzer to identify a current user state based on the at least one of the neurological data or the physiological data; and
a characteristic adjuster to modify a characteristic of the user device based on the current user state and a desired user state.

15. The system as defined in claim 14, wherein the characteristic adjuster is to dynamically modify the characteristic in real time or near real time to match changes in the user state.

16. The system as defined in claim 14, wherein the user state comprises at least one of alert, attentive, engaged, disengaged, drowsy, distracted, confused, asleep or nonresponsive.

17. The system as defined in claim 14, wherein the characteristic adjuster is to modify a characteristic of a user interface, and the user interface comprises at least one of an automatic teller machine interface, a checkout display, a mobile phone display, a computer display, an airport kiosk, a home appliance display, a vending machine display, a tablet display, a portable music player display, a phone display or a vehicle dashboard.

18. The system as defined in claim 14, wherein the characteristic adjuster is to modify a characteristic of a user interface by at least one of changing a font size, changing a hue, changing a screen brightness, changing a screen contrast, changing a volume, changing content, blocking a pop-up window, allowing a pop-up window, changing an amount of detail, changing a language, adding personalization or changing a size of an icon.

19. The system as defined in claim 14, wherein the neurological data includes one or more of functional magnetic resonance imaging data, electroencephalography data, magnetoencephalography data or optical imaging data.

20. The system as defined in claim 14 further comprising an alert, when the adjuster is to modify the characteristic by triggering the alert.

21. The system as defined in claim 14, wherein the analyzer is to re-identify the user state after the characteristic adjuster modifies the characteristics to determine an effectiveness of the modification.

22. The system as defined in claim 14, wherein the sensor is integrated in the user device measure at least one of the neurological or physiological data while the user operates the user device.

23. The system as defined in claim 14, wherein the current user state is the desired user state and the characteristic adjuster is to modify the characteristic to maintain the user in the current user state.

24. The system as defined in claim 14, wherein the current user state is not the desired user state and the characteristic adjuster is to modify the characteristic to change the user state.

25. A tangible machine readable medium storing instructions thereon which, when executed, cause a machine to at least:
collect at least one of neurological data or physiological data of a user interacting with the user device;
identify a current user state based on the at least one of the neurological data or the physiological data; and
modify a characteristic of the user device based on the current user state and a desired user state.

26. The machine readable media as defined in claim 25, wherein the instructions further cause a machine to dynamically modify the characteristic in real time or near real time to match changes in the user state.

27. The machine readable media as defined in claim 25, wherein the user state comprises at least one of alert, attentive, engaged, disengaged, drowsy, distracted, confused, asleep or nonresponsive.

28. The machine readable media as defined in claim 25, wherein the characteristic is a characteristic of a user interface, and the user interface comprises at least one of an automatic teller machine interface, a checkout display, a mobile phone display, a computer display, an airport kiosk, a home appliance display, a vending machine display, a tablet display, a portable music player display, a phone display or a vehicle dashboard.

29. The machine readable media as defined in claim 25 wherein the characteristic is a characteristic of a user interface and the instructions cause the machine to modify the characteristic by at least one of changing a font size, changing a hue, changing a screen brightness, changing a screen contrast, changing a volume, changing content, blocking a pop-up window, allowing a pop-up window, changing an amount of detail, changing a language, adding personalization or changing a size of an icon.

30. The machine readable media as defined in claim 25, wherein the neurological data includes one or more of functional magnetic resonance imaging data, electroencephalography data, magnetoencephalography data or optical imaging data.

31. The machine readable media as defined in claim 25, wherein the instructions cause the machine to modify the characteristic by activating an alert.

32. The machine readable media as defined in claim 25, wherein the instructions further cause the machine to re-identify the user state after modifying the characteristic to determine an effectiveness of the modification.

33. The machine readable media as defined in claim 25, wherein the instructions further cause the machine to collect the neurological data or the physiological data with a sensor of the user device while the user operates the user device.

34. The machine readable media as defined in claim 25, wherein the current user state is the desired user state and the instructions further cause the machine to modify the characteristic to maintain the user in the current user state.
